(19) United States
(12) Patent Application Publication
(10) Pub. No.: US 2011/0207215 A1
(43) Pub. Date: Aug. 25, 2011

(54) CELL STORAGE METHOD AND CELL TRANSPORT METHOD

(75) Inventors: Yoko Itchoda, Ibaraki (JP); Go Tazaki, Ibaraki (JP); Motohiro Fukuda, Ibaraki (JP); Hitoshi Tsuruta, Ibaraki (JP); Hideki Taniguchi, Kanagawa (JP)

(73) Assignees: KURARAY CO., LTD., Kurashiki-shi (JP); PUBLIC UNIVERSITY CORP. YOKOHAMA CITY UNIVERSITY, Yokohama-shi (JP)

(21) Appl. No.: 13/125,871

(22) PCT Filed: Oct. 23, 2009
(86) PCT No.: PCT/JP2009/005618

§ 371 Date: Apr. 25, 2011

(30) Foreign Application Priority Data
Oct. 24, 2008 (JP) 2008-273846

(51) Int. Cl. C12N 5/071; C12N 5/0735; C12N 5/0793; C12N 5/077

(52) U.S. Cl. 435/368; 435/366; 435/370

(57) ABSTRACT

To provide a cell storage method to store living cells while maintaining the functions of the living cells. An aspect of the cell storage method is a method to store living cells (40) by using a cell culture chamber (20) including a plurality of the microchambers (11), the method including: culturing the living cells by being adhered to the surfaces of a plurality of micro spaces; and after the culturing, pouring the culture medium (50) into the cell culture chamber (20) so as to cover the plurality of microchambers (11), and storing the living cells. The living cells are adhered to a cell culture chamber of an appropriate size and cultured to form a three-dimensional structure. This enables maintaining the three-dimensional structure of the living cells and storage of the living cells while maintaining the functions thereof.

### TECHNICAL FIELD

The present invention relates to a cell storage method and a cell transport method to store living cells.

### BACKGROUND ART

A technique of using cells isolated from a tissue in testing or examination is an essential method in the biotechnology-related fields. It is widely used in diagnosing a disease or pathological condition, searching for a new drug and evaluating the efficacy of a drug, or in animal inspection, plant inspection, testing for environmental pollutants, and so on. Thus, cells and the like used in the biotechnology field have been greatly diversified.

The isolated cells are sometimes used immediately for testing, but in many cases, the cells are cultured in a culture dish or a test tube. Various examinations are carried out using the cultured cells. Cell lines in culture for use in cell culture tests are required to show drug susceptibility and toxic reaction that are similar to those obtained in a test performed in a living body, that is, a so-called in vivo test. In short, it is necessary to be able to construct an intercellular network regularly arranged on the surface of a cell culture chamber. Further, the cell lines in culture for use in cell culture tests are extremely expensive, so an improvement in survival rate and proliferation rate of cells is desired.

The cell culture tests measure the effect of a drug or the like to be evaluated, by changing its amount, concentration, and the like under the same conditions. For this reason, it is necessary that the cell culture chambers be identical in material, shape, and the like. As the cell culture chambers, a petri dish made of plastic, a petri dish made of glass, a glass plate fixed into a chamber, a well plate, and the like are generally used. Examples of the well plate include 6-well, 12-well, 48-well, and 96-well plates or petri dishes. In general, the size of the entire plate is substantially the same, and as the number of wells increases, the size of a single well decreases. A single well corresponds to a single culture dish. With the recent trend toward miniaturization, a 384-well plate having a number of culture dishes with a small diameter has also come to be used. Bottoms of these culture dishes have a flat plate shape, and each of the bottom surfaces is used as a culture surface.

However, the use of the conventional cell culture chamber for culturing tissue cells causes the cells to be thinned into a form with no orientation. Additionally, the cells are randomly arranged on the surface of the cell culture chamber, so intercellular networks cross each other in a complicated manner. This causes a problem of being incapable of reproducing cell functions in vivo. In liver cells, for example, a mass of liver cells in spheroid that is known to have a function of maintaining the liver functions is not formed.

To overcome the above problems, there are disclosed a method for immobilizing particular polymers on a culture surface having a flat plate shape (see Patent Literature 1), a method using a particular apparatus (see Patent Literature 2), a method for culturing in a polymer gel (see Patent Literature 3), and the like.

However, in the method described in Patent Literature 1, stable production is impossible, since the immobilization method is complicated. This causes a problem of an increase in cost. Further, in the method described in Patent Literature 2, even though the culturing method is complicated, the formation efficiency of cell masses is low. This causes a problem of an increase in cost, for example. The method described in Patent Literature 3 has problems that the size of each cell mass cannot be controlled, and the operation of bases is complicated, for example. Therefore, these methods are complicated and incapable of stable production, and the costs increase.

### CITATION LIST

### Patent Literature

Patent Literature 1: Japanese Patent No. 3177610
Patent Literature 2: Japanese Unexamined Patent Application Publication No. 7-79772
Patent Literature 3: Japanese Unexamined Patent Application Publication No. 8-308562

### SUMMARY OF INVENTION

### Technical Problem

It is preferable to store living cells, which are isolated from a living body, in the same environment as in a living body. For example, in the case of storing non-frozen liver cells, the liver cells are adhered to a planar culture plate to be filled with a culture medium, and are stored and keep warm (for example, at a temperature of 25 to 37° C.). However, when the cells are cultured in the flat plate, the environment is greatly different from that within a living body. This causes a problem of losing the original functions of the cells. There is another problem that the cell functions are deteriorated due to environmental factors during culture or storage, such as shaking, temperature, and CO₂ concentration (pH change in the culture medium).

Further, there is a concern that when the cells cultured by the method disclosed in Patent Literature 1 or 2 are stored, cell death is caused by cell detachment or breakup of a gel in case of shaking, or the like. Therefore, it has been difficult to transport the stored living cells while maintaining the three-dimensional structure of living cells and to use the living cells at distance from the place where the living cells are separated. On the other hand, there is a demand for storing and transporting the isolated cells without degrading the functions of the isolated cells. This is because the isolation of cells from a tissue and the testing and examination thereof are carried out in different institutions.

The present invention has been made to solve the above-mentioned problems, and therefore has an object to provide a cell storage method and a cell transport method for storing living cells while maintaining cell functions.

### Solution to Problem

An aspect of a cell storage method according to the present invention is a cell storage method which stores living cells by using a cell culture chamber including a plurality of micro spaces, the cell storage method including: culturing the living cells by being adhered to surfaces of the plurality of micro spaces; pouring a culture medium into the cell culture chamber so as to cover the plurality of micro spaces after the culturing; and sealing the cell culture chamber so as to prevent the culture medium from leaking from the cell culture chamber, and storing the living cells. The living cells are adhered to the surfaces of the micro spaces and are cultured using micro spaces suitable for culture to form a three-dimensional structure within each of the microchambers, and the three-dimensional structure is isolated by the microchambers. After that, the cell culture chamber is sealed and stored, thereby making it possible to store the living cells while maintaining the functions thereof.

Further, it is preferred that the cell culture chamber be maintained at a temperature equal to or higher than 4 C. ° and lower than 37 C. ° to store the living cells. Furthermore, it is preferred that the culturing be carried out so that a cell population of a three-dimensional structure which is adhered to the surfaces and formed within each of the micro spaces has a number of cells that are separated from other cells. The use of the micro spaces prevents the living cells cultured in each of the micro spaces from contacting the living cells within other micro spaces.

It is preferred that the plurality of micro spaces have a bottom area of 0.01 to 0.1 mm² and a depth of 25 to 150 μm. It is also preferred that the living cells be one of liver cells, beta cells of pancreas, cardiac muscle cells, nerve cells, skin epidermal cells, cartilage cells, bone cells, tissue stem cells, ES cells, and iPS cells. Alternatively, the living cells are preferably one of liver cells, beta cells of pancreas, cardiac muscle cells, nerve cells, skin epidermal cells, cartilage cells, and bone cells, which are differentiated from one of tissue stem cells, ES cells, and iPS cells.

It is preferred that after the culturing, a non-adhered cell be removed and then the culture medium be poured into the cell culture chamber. It is also preferred that the culturing of the living cells include further culturing the living cells to allow the living cells to grow, elongate, or aggregate after the living cells are adhered and cultured.

It is preferred that the living cells be seeded in the plurality of micro spaces at a cell seeding density of 1×10² to 1×10⁶ cells/cm², more preferably at a cell seeding density of 1×10⁴ to 1×10⁶ cells/cm². It is preferred that a cell mass having the living cells accumulated therein be formed in each of the plurality of micro spaces. It is preferred that the cell mass has a diameter of 30 to 200 μm.

It is more preferred to satisfy at least one of the following conditions: the culture medium includes at least one of blood serum; a growth factor, and a component of a blood and the cell culture chamber is sealed with a membrane which allows oxygen or carbon dioxide to permeate.

An aspect of a cell transport method according to the present invention is a cell transport method for transporting living cells to be stored in a cell culture chamber including a plurality of micro spaces. In the cell transport method, the living cells are first cultured by being adhered to surfaces of the plurality of micro spaces. After the culturing, the culture medium is poured into the cell culture chamber so as to cover the plurality of the micro spaces. After that, the cell culture chamber is sealed so as to prevent the culture medium from leaking from the cell culture chamber. Then, the sealed cell culture chamber is transported by using transportation means which is one of a vehicle, a ship, and an aircraft. According to this cell transport method, it is possible to transport the living cells while maintaining the functions thereof. Further, it is preferred that the cell culture chamber be maintained at a temperature equal to or higher than 4 C. ° and lower than 37 C. °.

### Advantageous Effects of Invention

According to the present invention, it is possible to provide a cell storage method and a cell transport method for storing living cells while maintaining cell functions.

### BRIEF DESCRIPTION OF THE DRAWINGS

FIG. 1 is a plane view showing a structure of a cell culture chamber according to an embodiment;

FIG. 2 is a cross-sectional view along the line II-II showing the structure of the cell culture chamber according to an embodiment;

FIG. 3 is a plane view showing another structure of a cell culture chamber according to an embodiment;

FIG. 4 is a cross-sectional view along the line IV-IV showing another structure of the cell culture chamber according to an embodiment;

FIG. 5 is a plane view showing still another structure of a cell culture chamber according to an embodiment;

FIG. 6 is a cross-sectional view along the line VI-VI showing still another structure of the cell culture chamber according to an embodiment;

FIG. 7 is a view showing an exemplary state in which cells are cultured and sealed using the cell culture chamber shown in FIGS. 5 and 6;

FIG. 8A is a photograph showing a result of Example 06; and

FIG. 8B is a photograph showing a result of Example 07.

### DESCRIPTION OF EMBODIMENTS

A cell storage method according to the present invention is a method for culturing living cells by being adhered to microchambers using a cell culture chamber which is suitable for culturing the living cells, forming three-dimensional structures in the microchambers, filling the cell culture chamber with a culture medium after the culturing, and storing it. The microchambers allow the living cells to form the three-dimensional structures and maintain the structures. Further, the microchambers allow the three-dimensional structures of the living cells formed in the microchambers to be separated from other three-dimensional structures of the living cells. Therefore, there is a need to use suitable microchambers to culture the living cells. A chamber as described below is used as the cell culture chamber for the storage, for example.

A cell culture chamber has a concave-convex pattern, i.e., a plurality of microchambers formed therein. This permits cells to grow in three dimensions, like in a living body, and also permits cells to be cultured in aggregated form with no variation in each microchamber. The height of side walls (convex portions) for partitioning the microchambers is optimized, thereby making it possible to culture aggregated living cells (for example, a mass of liver cells) exclusively within the microchambers. Note that the term “micro space” refers to a space formed by a microchamber, more specifically to a space formed by a concave-convex pattern formed on a plane surface. Hereinafter, the microchamber and the micro space are not particularly distinguished from each other.

The dimensions of the microchambers each surrounded by the side walls have to be set within the optimum range for culturing cells. If the bottom area of each microchamber is too large, cells are thinly spread and fail to form a three-dimensional structure, as in the culture on a flat plate. If, on the other hand, the bottom area of each microchamber is too small, it cannot accommodate cells. Accordingly, the dimensions of the space structure are preferably in a range capable of containing one or a plurality of cells according to cell species to be cultured. In the case of forming the mass of liver cells in which a plurality of cells is accumulated, the dimensions are preferably in a range capable of containing the mass of liver cells.

The height of each side wall has to be set within the optimum range for preventing the cells cultured in the microchambers from moving to the adjacent microchambers. If the height of each side wall is too low, the cells run on the side wall, and thus such side wall is unsuitable for culture. If the height of each side wall is too high, the production thereof is difficult and material diffusion becomes difficult, leading to a deterioration of the culture environment. Therefore, the height of each side wall is preferably in the range capable of continuously and stably culturing cells, which are arranged in the microchambers according to cell species, within the microchambers.

In addition, openings are formed in the side walls to obtain a structure in which the plurality of microchambers communicates with each other, thereby making it possible to supply oxygen and nutrients to cells and remove waste products from the cells effectively. Note that the height of the side walls, the dimensions of the microchambers, and the width of the openings are appropriately set according to cell species to be cultured, thereby enabling application to various culture systems.

In this specification, the term “living cells” refers to cells (primary cultured cells) which are isolated from a living body tissue and which are not passaged. The living cells include two kinds of cells, frozen cells and fresh cells. The living cells also include cell lines, other ES cells (Embryonic Stem cells), and so on. The term “fresh cells” refers to primary cultured cells which are not frozen.

### Embodiment

Hereinafter, an embodiment of the present invention is described. However, the present invention is not limited to the following embodiment. Further, to clarify the explanation, the following description and the drawings are appropriately simplified.

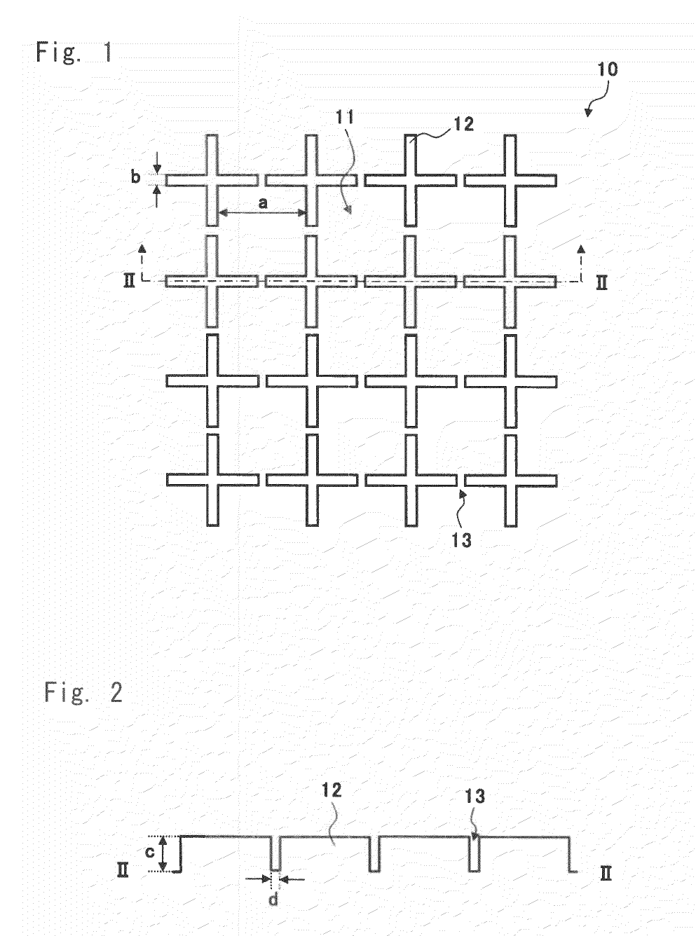

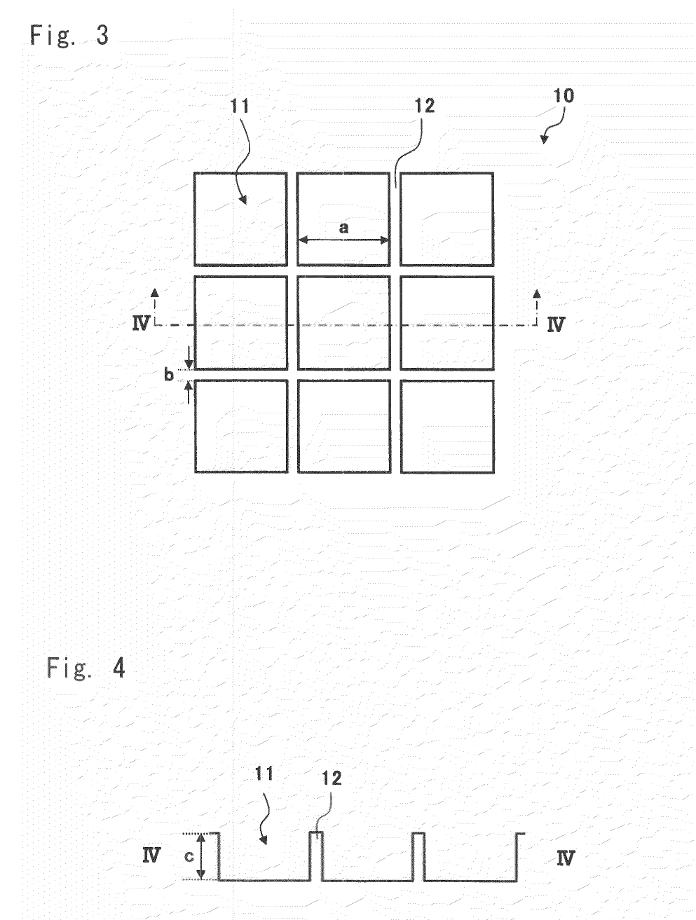

First, a cell culture chamber for use in a cell storage method according to an embodiment will be described, and subsequently, the cell storage method will be described. To begin with, an exemplary structure of the cell culture chamber will be described with reference to FIGS. 1 and 2. FIG. 1 is a plane view showing the structure of the cell culture chamber according to this embodiment, and FIG. 2 is a cross-sectional view along the line II-II in FIG. 1. As shown in FIG. 1, a cell culture chamber 10 includes microchambers 11, side walls 12, and openings 13. The plurality of side walls 12 is formed in a net shape on the culture surface of the cell culture chamber 10, and spaces surrounded by the side walls 12 serve as the microchambers 11. Additionally, each of the openings 13 is formed at a central portion of each side of the side walls 12 which are formed on four sides of each of the microchambers 11.

FIG. 1 shows a width “a” of the bottom of each of the microchambers 11, a width “b” and a height “c” of each of the side walls 12 for partitioning the microchambers 11, and a width “d” of each of the openings 13 for allowing communication between the microchambers 11 adjacent to each other. The term “bottom area” of the present invention refers to a projected area which is formed when parallel light is irradiated to the bottom of the chamber from above in the direction perpendicular to the horizontal plane of the microchamber opening (the same plane as the top surfaces of the side walls 12). For example, if the bottom of the microchamber is U-shaped, the bottom area has a shape formed by projecting parallel light incident on the bottom from above in the direction perpendicular to the opening plane. In the case of a circle or an ellipse, a major axis of a projected bottom is a distance between intersections of a long axis which runs through the center of gravity thereof and the circumference, and a minor axis of the projected bottom is a distance between intersections of a short axis which runs through the center of gravity thereof and the circumference. In the case of a polygon, the major axis and the minor axis of the projected bottom respectively correspond to a long axis and a short axis of an extrapolated circle or an extrapolated ellipse which is set so as to minimize the difference between areas of the polygon and the extrapolated circle or the extrapolated ellipse and which runs through all vertexes of the polygon. If an extrapolated circle or an extrapolated ellipse which runs through all vertexes of the polygon cannot be traced, the major axis and the minor axis respectively correspond to a long axis and a short axis of an approximate circle or an approximate ellipse which runs through the largest number of vertexes.

The bottom shape of each of the microchambers 11 is not particularly limited, and various shapes other than a square, a circle, and a polygon can be employed. In cell culture for reproducing a liver function in vivo, the bottom area is preferably 0.01 mm² to 0.1 mm². In this case, the major axis of the bottom is preferably 1 to 1.5 times the minor axis thereof. An isotropic shape is more preferably used. If a square is employed, for example, in the case of forming a mass of liver cells having an equivalent diameter of 100 μm, the length of one side thereof is preferably 100 μm to 300 μm.

An angle formed between the horizontal plane and the side walls 12 of each of the microchambers 11 should be set to an angle at which cells are prevented from running on the microchambers. Accordingly, 50% or more of an upper portion of a side surface preferably has an angle of 80° to 90°, and more preferably, 85° to 90°.

The height “c” of each of the side walls 12 may be arbitrarily set as long as the cells cultured in the microchambers 11 are prevented from running on and moving to the adjacent microchamber 11. In the case of forming a mass of liver cells having an equivalent diameter of 100 μm, the height “c” is preferably 50 μm to 150 μm, for example.

The width “d” of each of the openings 13 for allowing communication between the microchambers 11 adjacent to each other is preferably set to a width in which cells are prevented from moving from the microchamber 11, in which the cultured cell is first seeded, to the adjacent microchamber 11. When the equivalent diameter of the cultured cell is 20 μm, for example, the width is preferably 5 to 15 μm. Note that the openings 13 are not necessarily formed. As shown in FIGS. 3 and 4, the four sides of each of the microchambers 11 may be entirely surrounded by the side walls 12. Here, FIG. 3 is a plane view showing another structure of the cell culture chamber according to this embodiment, and FIG. 4 is a cross-sectional view along the line IV-IV in FIG. 3.

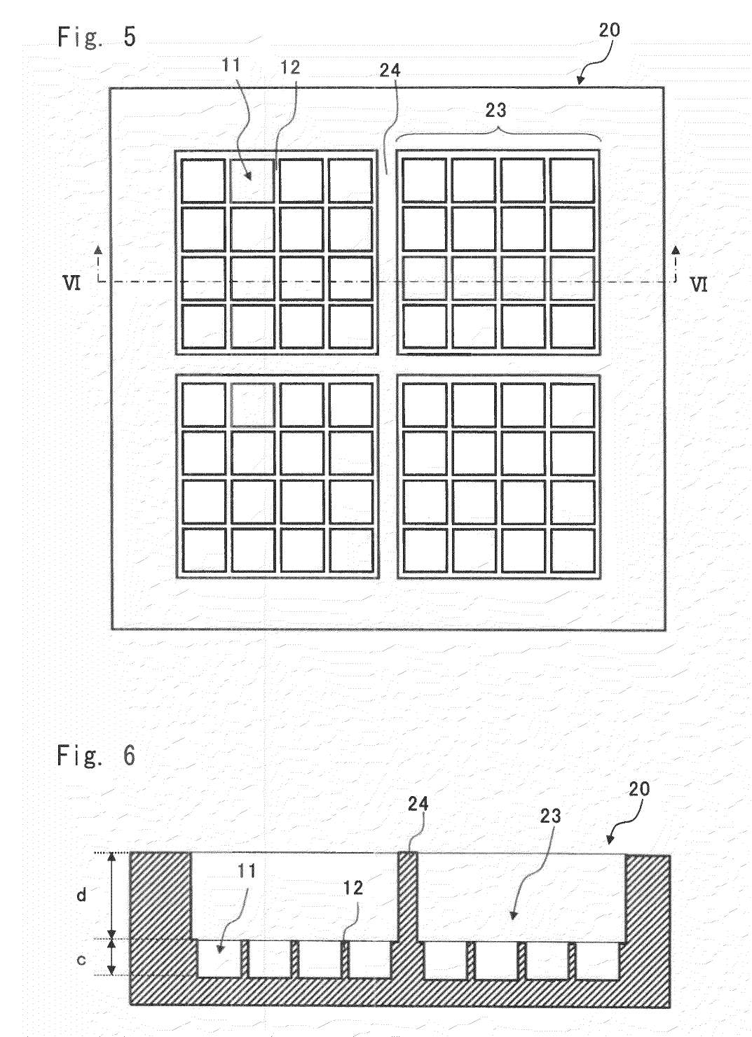

As shown in FIGS. 5 and 6, the cell culture chamber according to this embodiment may have partitioned spots each made up of a given numbers of microchambers. Here, FIG. 5 is a plane view showing still another structure of the cell culture chamber according to this embodiment, and FIG. 6 is a cross-sectional view along the line VI-VI in FIG. 5. FIGS. 5 and 6 show an example using the structure of the microchamber shown in FIGS. 3 and 4. FIG. 5 shows side walls 24 to partition the plurality of the microchambers, and partitioned spots 23. The height “d” of each of the side walls 24 may be arbitrarily set to satisfy a capacity for storing a supernatant fluid such as culture solution or reaction solution. Since the side walls 24 are provided, different culture mediums can be used in each of the spots 23. Though FIGS. 5 and 6 show an exemplary structure including the side walls 24, a structure without the side walls 24 may also be employed.

A method for forming the concave-convex pattern on the cell culture chamber is not particularly limited, but methods such as transfer molding using a mold, three-dimensional stereolithography, precision machining, wet etching, dry etching, laser processing, and electrical discharge machining may be employed. It is preferable to appropriately select these production methods in view of the intended use, required processing accuracy, costs, and the like of the cell culture chamber.

As a specific example of the transfer molding method using a mold, a method for forming the concave-convex pattern by resin molding using a metal structure as a mold may be employed. This method is preferred because it is capable of reproducing the shape of the metal structure on a resin as the concave-convex pattern with a high transcription rate, and because the raw material cost can be reduced by using a general-purpose resin material. Such a method using a mold of a metal structure is superior in terms of low cost and achieving satisfactorily high dimensional accuracy.

As methods of producing the metal structure, for example, plating treatment, precision machining, wet etching, dry etching, laser processing, and electrical discharge machining on a resist pattern produced by photolithography or a resin pattern produced by three-dimensional stereolithography may be employed. The methods may be appropriately selected in view of the intended use, required processing accuracy, costs, and the like.

As methods of forming the concave-convex pattern on a resin using the metal structure, which is obtained as described above, as a mold, injection molding, press molding, monomer casting, solvent casting, hot embossing, or roll transfer by extrusion molding may be employed, for example. It is preferable to employ injection molding in view of its productivity and transcription property.

Materials for forming a cell culture chamber are not particularly limited as long as the materials have self-supporting properties. For example, synthetic resin, silicon, or glass may be employed. A transparent synthetic resin is preferably used as a material in view of costs and cell visibility under microscopical observation. Examples of the transparent synthetic resin include acrylic resins such as polymethylmethacrylate or methyl methacrylate-styrene copolymer, styrene resin such as polystyrene, olefin resin such as cycloolefin, ester resins such as polyethylene terephthalate and polylactic acid, silicone resin such as polydimethylsiloxane, and polycarbonate resin. These resins may contain various additives such as colorant, dispersing agent, and thickening agent, unless the transparency is impaired.

In the cell culture chamber, surface treatment may be performed on the surface side of the concave-convex pattern and a modified layer and/or a coating layer may be formed for the purpose of improving the hydrophilic properties, biocompatibility, cellular affinity, and the like of the chamber surface. A method for forming the modified layer is not particularly limited unless a method with which the self-supporting properties are impaired and a method causing extreme surface roughness of 100 μm or more are employed. Methods, for example, chemical treatment, solvent treatment, chemical treatment such as introduction of a graft polymer by surface graft polymerization, physical treatment such as corona discharge, ozone treatment, or plasma treatment may be employed. In addition, though a method for forming the coating layer is not particularly limited, methods, for example, dry coating such as sputtering or vapor deposition and wet coating such as inorganic material coating or polymer coating may be employed. In order to pour a culture solution without mixing air bubbles therein, it is desirable to impart the hydrophilic properties to the surface of the concave-convex pattern. As a method for forming a uniform hydrophilic membrane, inorganic vapor deposition is preferably employed.

When the cellular affinity is taken into consideration, it is more preferable to coat cytophilic proteins such as collagen and fibronectin. In order to uniformly coat a collagen aqueous solution or the like, it is preferable to perform the coating after the above-mentioned hydrophilic membrane is formed. In hepatocyte cultures, in general, it is desirable to culture cells on an extracellular matrix surface by replicating the in vivo environment. Accordingly, it is particularly preferable to dispose an organic film made of extracellular matrix suitable for cultured cells after an inorganic hydrophilic membrane is uniformly formed as described above.

In a cell culture method using the cell culture chamber described above, an appropriate number of cells need to be seeded so that the cells are arranged exclusively within the microchambers for culturing cells, and morphologies and functions similar to those of the living body are developed within the space. A cell seeding density of 1.0×10² to 1.0×10⁶ cells/cm² is preferably used and a cell seeding density of 1.0×10⁴ to 1.0×10⁶ cells/cm² is more preferably used. When each microchamber is a square which is 200 μm on a side, for example, a cell seeding density of 5.0×10⁴ to 5.0×10⁵ cells/cm² is preferably used. Under such conditions, a mass of liver cells having a diameter of 30 to 200 μm can be obtained.

Subsequently, the cell storage method according to this embodiment will be described. The cell storage method is a method to culture living cells by using the cell culture chamber including the plurality of culture microchambers described above and to store it. Specifically, first, a culture process is carried out in which the living cells are adhered to the surfaces of the plurality of culture microchambers of the cell culture chamber to culture the cells. Next, a storage preparation process is carried out in which a culture medium (culture solution) is poured into the cell culture chamber so as to cover the plurality of microchambers to prepare for the storage. The cell culture chamber prepared as described above is stored (storage process). Each process will be described in more detail below.

First, the culture process will be described. Adhesive cells are used as the living cells. The living cells are adhered to the surfaces of the microchambers, thereby preventing the living cells from colliding with the surfaces of the microchambers, or flowing out from the microchambers, for example, during the storage and transportation. This inhibits loss of the functions of the cultured living cells, and prevents the living cells from drying due to being exposed from the culture medium and from degradation of the cell functions due to the dry state.

Parenchymal cells are used as the living cells to be cultured. Specifically, any one of liver cells, beta cells of pancreas, cardiac muscle cells, nerve cells, skin epidermal cells, cartilage cells, bone cells, tissue stem cells, ES cells, and iPS cells (induced pluripotent stem cells) are used. Alternatively, any one of liver cells, beta cells of pancreas, cardiac muscle cells, nerve cells, skin epidermal cells, cartilage cells, and bone cells, which are differentiated from one of tissue stem cells, ES cells, and iPS cells.

The culture process includes (1) a step of adhering cells, (2) a step of removing non-adhered cells, and (3) a step of further culturing cells to allow the cells to grow, elongate, or aggregate. Removal of the non-adhered cells makes it possible to remove waste products and to prevent the culture medium from being contaminated. Additionally, the living cells are cultured by culture growth or the like until the mass of living cells reaches a desired size. For example, the living cells are allowed to grow, elongate, and aggregate until the diameter of a cellular aggregate reaches 30 to 200 μm. Each of the microchambers is designed to be large enough to contain the desired cell mass. As just described, in the culture of the living cells, the cells are cultured so that the living cells are adhered (seeded) onto the surfaces of the plurality of the microchambers, and the cell population of the three-dimensional structure formed within the space of each of the microchambers has a number of cells that are isolated from other cells.

According to the steps as described above, a cell population which corresponds to a number of cells of a single three-dimensional structure is formed within each microchamber and is isolated form other cells. Here, the cell population (cell mass) formed within each microchamber corresponds to a single three-dimensional structure. The living cells are isolated by the walls of the microchambers having a height greater than the size of the living cells and the living cells are adhered to the microchambers. Therefore, the living cells are prevented from coming into contact with adjacent three-dimensional structures. This allows the living cells to form or maintain a uniform three-dimensional structure even under a non-static circumstance (such as a transported state).

Next, the storage preparation process will be described. The amount of culture medium to be poured is determined so as to prevent the cultured living cells from drying or contacting outside air. The culture medium for use in the storage is a medium containing nutrient components, for example, a blood serum or a component of blood, such as a growth factor. Components contained in the culture medium are determined depending on the living cells to be stored.

Further, the cell culture chamber is sealed by seal means so as to prevent the culture medium from leaking from the cell culture chamber. The cell culture chamber has a structure in which the plurality of microchambers is formed on, for example, a petri dish, a plate, or a flask having an opening portion. In the sealing process, the opening portion formed in the upper surface of the cell culture chamber is covered by the seal means, such as a film or a cap, and the living cells are stored in the state of being separated from the outside. The seal means may be made of a material that blocks a flow of liquid or gas, or a material that allows carbon dioxide or oxygen to permeate. This may be selected depending on the living cells to be stored or a culture state. Additionally, in the case of transporting the living cells, it is necessary that the culture medium is sealed so as not to be leaking from the chamber. On the other hand, during a storage in a static state, it may be allowable for closing a lid so as to prevent from drying the culture medium, or for storing with the opening portion opened. Depending on the stored state of the living cells, the cell culture chamber is not necessarily sealed.

Finally, the storage process will be described. In the storage process, it is preferable that the cell culture chamber be adjusted to a temperature within a temperature range equal to or higher than 4° C. and lower than 37° C., preferably a temperature range equal to or higher than 6° C. and lower than 25° C., and more preferably a temperature range from 10° C. to 20° C. Additionally, the storage process also includes a period for transporting the cell culture chamber which is produced and sealed in the store preparation process. It is preferable to carry out temperature adjustment also during the transportation period.

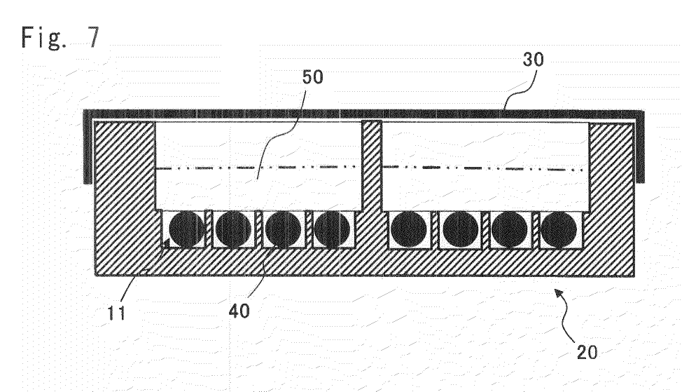

FIG. 7 shows an example of a state of the cell culture chamber after the storage preparation process. FIG. 7 shows an example where the culture process and the storage preparation process are carried out by using the cell culture chamber shown in FIGS. 5 and 6 and living cells 40 and seal means 30 are added to the sectional view shown in FIG. 6. Specifically, the living cells 40 are adhered to each of the microchambers 11 within a cell culture chamber 20 and cultured. In FIG. 7, the living cells 40 are simply represented as a black circles, and a culture medium 50 is filled up to a level indicated by a two-dot chain line. The seal means 30 seals the cell culture chamber 20. The amount of culture medium shown in FIG. 7 is an example. Alternatively, an amount of culture medium corresponding to on-third of the volume of the chamber may be poured, or the culture medium may be poured until there is no airspace between the seal means 30 and the culture medium. The volume of the culture medium is arbitrarily selected depending on the kinds of living cells, culture state, or the like.

As described above, according to an aspect of the embodiment, it is possible to allow the living cells to form the three-dimensional structures adhered to a culture base made of hard plastic, without using any particular polymers, and to be stored.

It is known that when living cells form a three-dimensional structure, the functions of the cells are improved. Therefore, it is possible to store the living cells while maintaining the functions thereof, after the living cells are cultured.

Further, when the cells are transported by transportation means, such as a vehicle, a ship, or a an aircraft, there is a concern that the functions of cells are further deteriorated due to environmental factors during transportation, such as shaking, temperature, and CO₂ concentration (pH change in the culture medium), and that cell death is caused by cell ablation or breakup of a gel due to shaking occurring during transportation. As to these problems, it is possible to transport living cells while maintaining the functions of the living cells, by applying an aspect of the present invention.

For example, the present invention is applicable when fresh liver cells isolated from a living body are transported. Specifically, it is considered that the fresh liver cells isolated from a living body can live for only a predetermined period of time (60 hours). Even in such a case, it is to be expected that the three-dimensional structures of fresh liver cells can be maintained, and that the cells can be transported while maintaining the functions. Further, an aspect of the present invention is applicable when the fresh liver cells isolated from a living body are transported in a frozen state. That is, after melting the frozen cells, it is to be expected that the cells can be stored or transported in a non-frozen state by employing the method of the present invention. Furthermore, when the three-dimensional structures of the living cells were transported by using polymers, such as gel, there were cases where the three-dimensional structures of the living cells was broken due to breakup of gel from the living cells or a change in shape of the gel. It is to be expected that the three-dimensional structures can be maintained, and that the living cells can be transported while maintaining the functions thereof, by applying an aspect of the present invention.

### Example

Next, examples of the cell culture method according to the present invention are described hereinafter; however, the present invention is not limited to those examples.

### Example 01

Example showing a case where cells are stored using a cell culture chamber including a plurality of micro spaces and a case where cells are stored using a flat plate.

### Culture Chamber

### Example 01

A pattern which has the shape of the concave-convex pattern as shown in FIGS. 3 and 4 and which has dimensions of a=200 μm, b=20 μm, and c=50 μm was produced by photolithography, and Ni electrolytic plating was carried out to obtain a mold having a corresponding concave-convex shape. The concave-convex pattern shape was transcribed on polystyrene by hot embossing with the mold, and a resin base material having the above-mentioned dimensions was produced. A film was produced by forming a silicon dioxide film with a thickness of 100 nm on the surface of the resin base material by vacuum deposition. The film was attached to a 24-hole plate having no culture bottom and made of polystyrene, and γ-ray sterilization was carried out to produce the culture chamber including the plurality of 24-well micro spaces for use in a storage test.

### Comparative Example 01

A commercially available (Falcon (Registered Trademark) available from Becton, Dickinson and Company) γ-ray sterilized flat 24-well culture plate was used for the storage test.

### (Cell Culture)

Human liver carcinoma cell line HepG2 cells (Japan Health Sciences Foundation, Health Science Research Resources Bank resource number JCRB1054) were grown to a predetermined number of cells in an incubator at 37° C. and 5% CO₂ using a culture flask (manufactured by Corning Incorporated). A DMEM culture medium containing 10% fetal bovine serum (manufactured by GIBCO) was used as the culture medium. The grown cells were detached from the culture bottoms using a 0.25% trypsin solution and collected by a centrifugal separation method. The collected cells were adjusted to a cell concentration of 4×10⁵ cells/ml by using a DMEM culture solution containing 10% FBS which was added to each well by 500 μl. After that, the cells were cultured in the incubator at 37° C. and 5% CO₂ for three days.

### (Storage Test)

The cells were cultured in the incubator at 37° C. and 5% CO₂ using the culture chambers shown in Example 01 and Comparative Example 01. After that, the culture medium was vacuumed and the DMEM culture medium containing 10% FBS was newly added to each well by 500 μl. After the culture chamber was sealed with a plastic film, the culture chamber was put into an incubator at a temperature of 37° C. and stored for 24 hours.

### (Analysis: Measurement of Albumin Secretion)

After the storage for 24 hours, the culture medium was vacuumed and the DMEM culture medium containing 10% FBS was added to each well. Then, the cells were cultured in the incubator at 37° C. and 5% CO₂ for two days. Cultured supernatant was collected, and a human albumin secretion for two days was measured by human albumin analysis ELISA kits (manufactured by Bethyl Laboratories, Inc.).

### (Result)

Table 1 shows the result of measurement of albumin secretion. The secretion shown in Example 01 is three times the secretion of Comparative Example 01.

TABLE 1

| | Albumin Secretion [pg/ml/2 days] |
|---|---|
| Example 01 | 360 |
| Comparative Example 01 | 120 |

### Examples 02 to 05

### Examples Given in Terms of Storage Temperature

### (Culture Chamber)

A pattern which has the shape of the concave-convex pattern as shown in FIGS. 3 and 4 and which has dimensions of a=200 μm, b=20 μm, and c=50 μm was produced by photolithography, and Ni electrolytic plating was carried out to obtain a mold having a corresponding concave-convex shape. The concave-convex pattern shape was transcribed on polystyrene by hot embossing with the mold, and a resin base material having the above-mentioned dimensions was produced. A film was produced by forming a silicon dioxide film with a thickness of 100 nm on the surface of the resin base material by vacuum deposition. The film was attached to a 24-hole plate having no culture bottom and made of polystyrene, and γ-ray sterilization was carried out to produce the culture chamber (culture plate) including the plurality of 24-well micro spaces for use in a storage test.

### (Cell Culture)

Human liver carcinoma cell line HepG2 cells (Japan Health Sciences Foundation, Health Science Research Resources Bank resource number JCRB1054) were grown to a predetermined number of cells in an incubator at 37° C. and 5% CO₂ using a culture flask (manufactured by Corning Incorporated). A DMEM culture medium containing 10% fetal bovine serum (manufactured by GIBCO) was used as the culture medium. The grown cells were detached from the culture bottoms using a 0.25% trypsin solution and collected by a centrifugal separation method. The collected cells were adjusted to a cell concentration of 4×10⁵ cells/ml by using a DMEM culture solution containing 10% FBS which was added to each well of the culture chamber by 500 μl. After that, the cells were cultured in the incubator at 37° C. and 5% CO₂ for three days.

### (Storage Test)

After culturing the cells for three days, the culture medium was vacuumed and the DMEM culture medium containing 10% FBS was newly added to each well 500 μl. The culture chamber was sealed with a plastic film and was then stored by the storage method according to the following Examples. Temperature inside the container was measured by a temperature sensor, and it was checked whether the temperatures were maintained at a target temperature.

### Example 02

A storage container (manufactured by Hitachi Transport System, Ltd.) containing a lagging material for keeping the container at 18° C. was used. The culture chamber was stored in the storage container for 24 hours. The temperature inside the storage container during this period was 18° C.±0.5° C.

### Example 03

A storage container (manufactured by Hitachi Transport System, Ltd.) containing a lagging material for keeping the container at 6° C. was used. The culture chamber was stored in the storage container for 24 hours. The temperature inside the storage container during this period was 6° C.±0.5° C.

### Example 04

The culture chamber was put on a container made of Styrofoam and containing ice and stored for 24 hours. The temperature of the bottom of the culture chamber during this period was 0° C.±0.5° C.

### Example 05

The culture chamber was put into the incubator at 37° C. and stored for 24 hours.

### (Analysis)

After the storage for 24 hours, the culture medium was vacuumed and the DMEM culture medium containing 10% FBS was added to each well. Then, the cells were cultured in the incubator at 37° C. and 5% CO₂ for 24 hours. After that, observation was carried out using an inverted microscope to count the number of cell masses having a size of 30 to 200 μm in a given field. The cell mass formation rate was calculated by the following equation. The cell mass formation rate before the storage was also calculated in the same manner.

$$Cell Mass Formation Rate = \frac{( \begin{matrix}The number of micro spaces in one field \\ in which a cell mass is formed\end{matrix} )}{( The number of micro spaces in one field )} \times 100 [ \% ] \qquad ( Equation 1 )$$

Where, in Equation 1, the term “The number of micro spaces in one field” refers to the number of micro spaces (microchambers) existing in one field (a given range of field). The term “The number of micro spaces in one field in which a cell mass is formed” refers to the number of micro spaces, in which a cell mass having a size of 30 to 200 μm is formed, among the micro spaces in one field.

### (Result)

Table 2 shows the cell mass formation rate. The cell mass formation rate before the storage was 80 to 100%. In Example 02 (18° C.), the morphology before the storage was almost completely maintained. In Example 03 (6° C.), the cell mass formation rate decreases, but 60% to 80% of the morphology was maintained. In Example 04 (0° C.), the cell mass formation rate was as low as 30%, and the morphology was hardly maintained. In Example 05 (37° C.), the cell mass formation rate was 25%, which was lowest, and the morphology was hardly maintained.

From this result, it turns out that the storage temperature is preferably from 10° C. to 20° C., more preferable 18° C.

TABLE 2

| | Example | Example | Example | Example |
|---|---|---|---|---|
| | 02 | 03 | 04 | 05 |
| Cell mass formation rate | 98 | 60 | 31 | 25 |
| (%) | | | | |

### Examples 06 and 07>

### Examples Given in Terms of the Cell Density

### (Culture Chamber)

In Examples 06 and 07, the same culture chamber of Examples 02 to 05 was used.

### (Cell Culture)

Human liver carcinoma cell line HepG2 cells (Japan Health Sciences Foundation, Health Science Research Resources Bank resource number JCRB1054) were grown to a predetermined number of cells in an incubator at 37° C. and 5% CO₂ using a culture flask (manufactured by Corning Incorporated). A DMEM culture medium containing 10% fetal bovine serum (manufactured by GIBCO) was used as the culture medium. The grown cells were detached from the culture bottoms using a 0.25% trypsin solution and collected by a centrifugal separation method. The collected cells were adjusted to cell concentration described in Examples by using a DMEM culture solution containing 10% FBS which was added to each well of the culture chamber by 500 After that, the cells were cultured in the incubator at 37° C. and 5% CO₂ for three days.

### Example 06

When the cells were seeded, the cell concentration was adjusted to 4×10⁵ cells/ml, and the cells were stored under the storage conditions described below.

### Example 07

When the cells were seeded, the cell concentration was adjusted to 40×10⁵ cells/ml, and the cells were stored under the storage conditions described below.

### (Storage Conditions)

After culture for three days, the culture medium was vacuumed and the DMEM culture medium containing 10% FBS was added to each well by 500 μl. The culture chamber was sealed with a plastic film. After that, the culture chamber was put into a storage container (manufactured by Hitachi Transport System, Ltd.) containing a lagging material for keeping the container at 18° C., and was stored for 24 hours.

### (Analysis)

### (Albumin Analysis)

After the storage for 24 hours, the culture medium was vacuumed and the DMEM culture medium containing 10% FBS was added to each well. Then, the cells were cultured in the incubator at 37° C. and 5% CO₂ for two days. Cultured supernatant was collected, and a human albumin secretion for two days (ng/ml/2 days) was measured by human albumin analysis ELISA kits (manufactured by Bethyl Laboratories, Inc.). Next, the cells were detached by using a 0.25% trypsin solution, the values of the living cells were calculated using trypan blue. Table 3 below shows values per 10⁵ cells as albumin secretion (pg/10⁵/2 days).

### (Morphology Observation)

After the storage for 24 hours, the culture medium was vacuumed and the DMEM culture medium containing 10% FBS was added to each well. Then, the cells were cultured in the incubator at 37° C. and 5% CO₂ for 24 hours in the incubator at 37° C. and 5% CO₂. After that, the morphology was observed by using an inverted microscope.

### (Result)

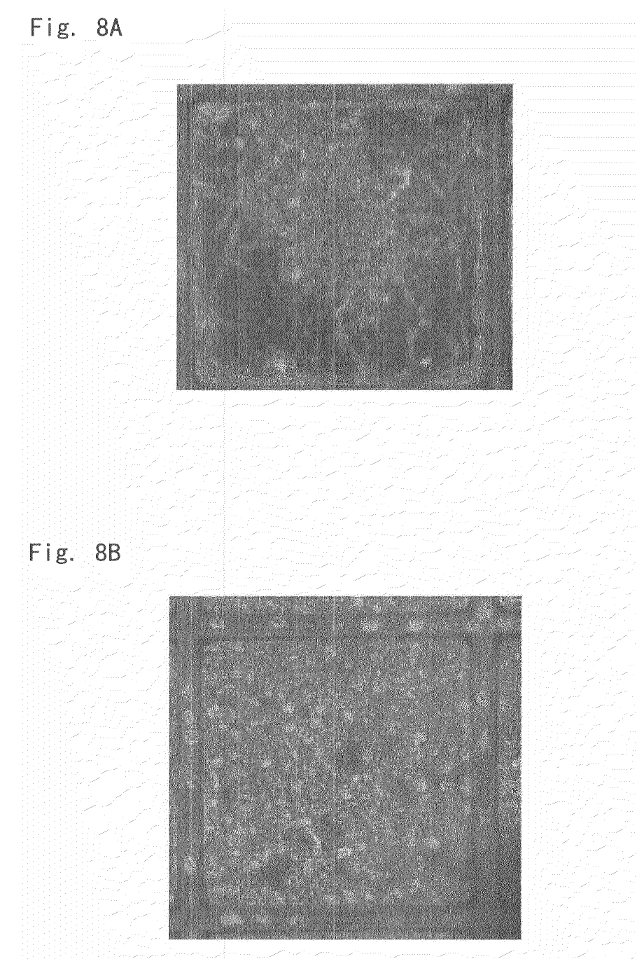

FIGS. 8A and 8B are photographs taken when the cells were cultured in the incubator at 37° C. and 5% CO₂ for 24 hours after the plate was extracted from the storage container.

In Example 06 (FIG. 8A), spherical cell masses were formed. On the other hand, in Example 07 (FIG. 8B), the cells were densely packed in each micro chamber to form cell masses, and dead cells aggregate outside the partitions. Table 3 shows albumin secretions. In terms of the albumin secretion, Example 06 shows a value as high as about 2.8 times that of Example 07.

TABLE 3

| | Albumin Secretion [pg/10⁵/2 days] |
|---|---|
| Example 06 | 177 |
| Example 07 | 62.5 |

Note that the present invention is not limited to above-described embodiments. The elements of the embodiments can be modified, added, or converted to the contents that can be easily thought of by those skilled in the art within the scope of the present invention.

### REFERENCE SIGNS LIST

10, 20 CELL CULTURE CHAMBER
11 MICROCHAMBER
12 SIDE WALL
13 OPENING
23 SPOT
24 SIDE WALL OF SPOT
30 SEAL MEANS
40 LIVING CELL
50 CULTURE MEDIUM

1. A cell storage method for storing living cells with a cell culture chamber comprising a plurality of micro spaces, the method comprising:
culturing the living cells by adhering the living cells to surfaces of the plurality of micro spaces;
pouring a culture medium into the cell culture chamber so as to cover the plurality of micro spaces after the culturing; and
sealing the cell culture chamber so as to prevent the culture medium from leaking from the cell culture chamber, and thereby storing the living cells.

2. The method of claim 1, wherein
the cell culture chamber is maintained at a temperature equal to or higher than 4 C. ° and lower than 37 C. ° to store the living cells.

3. The method of claim 1, wherein
the culturing is carried out so that a cell population of a three-dimensional structure which is adhered to the surfaces and formed within each of the micro spaces has a number of cells that are separated from other cells.

4. The method of claim 1, wherein
the plurality of micro spaces have a bottom area of 0.01 to 0.1 mm² and a depth of 25 to 150 μm.

5. The method of claim 1, wherein
the living cells are at least one selected from the group consisting of a liver cell, a pancreatic beta cell, a cardiac muscle cell, a nerve cell, a skin epidermal cell, a cartilage cell, a bone cell, a tissue stem cell, an ES cell, and an iPS cell.

6. The method of claim 1, further comprising
the living cells are at least one selected from the group consisting of a liver cell, a pancreatic beta cell, a cardiac muscle cell, a nerve cell, a skin epidermal cell, a cartilage cell, and a bone cell, which are differentiated from at least one selected from the group consisting of a tissue stem cell, an ES cell, and an iPS cell.

7. The method of claim 1, further comprising
after the culturing, removing a non-adhered cell, and then pouring the culture medium into the cell culture chamber.

8. The method of claim 1, wherein
the culturing of the living cells comprises further culturing the living cells to allow the living cells to grow, elongate, or aggregate after the living cells are adhered and cultured.

9. The method of claim 1, wherein
the living cells are seeded in the plurality of micro spaces at a cell seeding density of 1×10² to 1×10⁶ cells/cm².

10. The method of claim 1, wherein
a cell mass having the living cells accumulated therein is formed in each of the plurality of micro spaces.

11. The method of claim 9, wherein
the cell mass has a diameter of 30 to 200 μm.

12. The method of claim 1, wherein
the culture medium comprises at least one selected from the group consisting of a blood serum, a growth factor, and a component of blood.

13. The method of claim 1, wherein
the cell culture chamber is sealed with a membrane which allows oxygen or carbon dioxide to permeate.

14. A cell transport method for transporting living cells to be stored in a cell culture chamber comprising a plurality of micro spaces, the method comprising:
culturing the living cells by adhering the living cells to surfaces of the plurality of micro spaces;
pouring a culture medium into the cell culture chamber so as to cover the plurality of micro spaces after the culturing;
sealing the cell culture chamber so as to prevent the culture medium from leaking from the cell culture chamber; and
transporting the sealed cell culture chamber by at least one selected from the group consisting of a vehicle, a ship, and an aircraft.

15. The method of claim 14, wherein
the cell culture chamber is maintained at a temperature equal to or higher than 4 C. ° and lower than 37 C. °.

16. The method of claim 2, wherein
the culturing is carried out so that a cell population of a three-dimensional structure which is adhered to the surfaces and formed within each of the micro spaces has a number of cells that are separated from other cells.

17. The method of claim 16, wherein
the plurality of micro spaces have a bottom area of 0.01 to 0.1 mm² and a depth of 25 to 150 μm.

18. The method of claim 1, wherein
the living cells are one member selected from the group consisting of a liver cell, a pancreatic beta cell, a cardiac muscle cell, a nerve cell, a skin epidermal cell, a cartilage cell, a bone cell, a tissue stem cell, an ES cell, and an iPS cell.

19. The method of claim 1, further comprising
the living cells are one member selected from the group consisting of a liver cell, a pancreatic beta cell, a cardiac muscle cell, a nerve cell, a skin epidermal cell, a cartilage cell, and a bone cell, which are differentiated from one member selected from the group consisting of a tissue stem cell, an ES cell, and an iPS cell.

20. The method of claim 2, wherein
the plurality of micro spaces have a bottom area of 0.01 to 0.1 mm² and a depth of 25 to 150 μm.
